(19) United States
(12) Patent Application Publication
(10) Pub. No.: US 2019/0290113 A1
(43) Pub. Date: Sep. 26, 2019

(54) ENDOSCOPE APPARATUS AND MEDICAL IMAGING DEVICE

(71) Applicant: Sony Olympus Medical Solutions Inc., Tokyo (JP)

(72) Inventors: Sumihiro UCHIMURA, Tokyo (JP); Yuichi YAMADA, Tokyo (JP)

(73) Assignee: Sony Olympus Medical Solutions Inc., Tokyo (JP)

(21) Appl. No.: 16/273,227

(22) Filed: Feb. 12, 2019

(30) Foreign Application Priority Data
Mar. 22, 2018 (JP) 2018-054560

(51) Int. Cl. A61B 1/06; A61B 1/00; A61B 1/05

(52) U.S. Cl. CPC A61B 1/0676; A61B 1/00096; A61B 1/00163; G02B 23/2461; A61B 1/00009; A61B 1/00117; A61B 1/05

(57) ABSTRACT

An endoscope apparatus includes an insertion unit having at a distal end thereof a light-incident end portion that captures observation light from a subject, the insertion unit being insertable into an object to be examined; and an ultraviolet light source unit that emits ultraviolet light. The light-incident end portion is provided with an ultraviolet light absorption filter that generates heat by absorbing the ultraviolet light emitted from the ultraviolet light source unit.

### CROSS-REFERENCE TO RELATED APPLICATION(S)

The present application claims priority to and incorporates by reference the entire contents of Japanese Patent Application No. 2018-054560 filed in Japan on Mar. 22, 2018.

### BACKGROUND

The present disclosure relates to an endoscope apparatus and a medical imaging device.

In the past, endoscope apparatuses have been known which includes an endoscope having an insertion unit that is inserted into an object to be examined thereby to capture light from a subject, an imaging device having an imaging element that receives the light captured by the endoscope and converts the light into an electric signal, and an image processing device that generates a captured image based on the electric signal generated by the imaging device.

When the endoscope is inserted into the object to be examined, dew condensation occurs on the cover glass provided at the distal end portion of the insertion unit due to the temperature difference between the distal end portion and the body cavity. Since the occurrence of dew condensation causes the imaging field of view to become cloudy, there is a problem that a clearly captured image cannot be acquired. As a solution to this problem, a method of preventing dew condensation by providing a heating unit, such as a heating element, in the vicinity of the cover glass is mentioned (for example, Japanese Laid-open Patent Publication No. 2006-282, referred to as JP 2006-282 A, hereinafter). However, in order to reduce the invasion of the object to be examined, the diameter of the insertion unit is required to be reduced. Providing a heating unit in the vicinity of the cover glass causes an increase in the diameter of the insertion unit. In addition, among endoscopes, a rigid endoscope does not have an electric circuit in the insertion unit. For this reason, in the case of providing a heating unit on the cover glass, it is necessary to provide a circuit or the like for heating the heating unit.

As a technique for preventing dew condensation while suppressing the increase in the diameter of the insertion unit, a technique of providing a filter for cutting light in the infrared wavelength band (infrared light) on the cover glass and heating the filter with infrared light to prevent dew condensation has been proposed (for example, Japanese Laid-open Patent Publication No. H2-48628 A, referred to as JP H2-48628 A, hereinafter).

### SUMMARY

In recent years, in addition to normal observation using white light (visible light), infrared observation that is an observation using infrared light from a subject has been put to practical use in the endoscope apparatus. However, in the technique disclosed in JP H2-48628 A, since the filter for cutting infrared light is provided on the cover glass, the infrared light from the subject is cut by the filter and accordingly the infrared light is not guided to the imaging element.

The present disclosure has been made in view of the above, and is directed to an endoscope apparatus and a medical imaging device.

According to a first aspect of the present disclosure, an endoscope apparatus is provided which includes an insertion unit having at a distal end thereof a light-incident end portion that captures observation light from a subject, the insertion unit being insertable into an object to be examined; and an ultraviolet light source unit that emits ultraviolet light, wherein the light-incident end portion is provided with an ultraviolet light absorption filter that generates heat by absorbing the ultraviolet light emitted from the ultraviolet light source unit.

According to a second aspect of the present disclosure, there is provided a medical imaging device detachably connected to an endoscope including an insertion unit having at a distal end thereof a light-incident end portion that captures observation light from a subject, the insertion unit being insertable into an object to be examined, the light-incident end portion having an ultraviolet light absorption filter that generates heat by absorbing ultraviolet light. The medical imaging device includes an imaging unit that receives the observation light and generates an imaging signal; and at least one dichroic mirror provided in an optical path of the observation light, the at least one dichroic mirror being configured to guide, to the imaging unit, the ultraviolet light from the ultraviolet light source unit to the ultraviolet light absorption filter by one of transmission and reflection, and at least light having a wavelength in a visible region of the observation light to the imaging unit by the other one of transmission and reflection.

The above and other objects, features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating the schematic configuration of an endoscope apparatus according to a first embodiment of the present disclosure;

FIG. 2 is a block diagram illustrating the configuration of a camera head and a control device illustrated in FIG. 1;

FIG. 3 is a schematic diagram illustrating the configuration of an endoscope and a light source device according to the first embodiment of the present disclosure;

FIG. 4 is a schematic diagram illustrating the distal end configuration of the endoscope according to the first embodiment of the present disclosure;

FIG. 5 is a schematic diagram illustrating the configuration of an endoscope and a light source device according to a first modification of the first embodiment of the present disclosure;

FIG. 6 is a schematic diagram illustrating the distal end configuration of the endoscope according to the first modification of the first embodiment of the present disclosure;

FIG. 7 is a schematic diagram illustrating the configuration of an endoscope and a light source device according to a second modification of the first embodiment of the present disclosure;

FIG. 8 is a schematic diagram illustrating the configuration of an endoscope and a light source device according to a third modification of the first embodiment of the present disclosure;

FIG. 9 is a schematic diagram illustrating the configuration of an endoscope and a light source device according to a fourth modification of the first embodiment of the present disclosure;

FIG. 10 is a schematic diagram illustrating the configuration of the endoscope and the light source device according to the fourth modification of the first embodiment of the present disclosure;

FIG. 11 is a diagram illustrating the schematic configuration of an endoscope apparatus according to a second embodiment of the present disclosure;

FIG. 12 is a schematic diagram illustrating the configuration of an endoscope and a light source device according to the second embodiment of the present disclosure;

FIG. 13 is a schematic diagram illustrating the configuration of an endoscope and a light source device according to a first modification of the second embodiment of the present disclosure;

FIG. 14 is a schematic diagram illustrating the configuration of an endoscope and a light source device according to a second modification of the second embodiment of the present disclosure; and

FIG. 15 is a schematic diagram illustrating the distal end configuration of an endoscope according to another embodiment of the present disclosure.

### DETAILED DESCRIPTION

Hereinafter, modes (hereinafter, embodiments) for carrying out the present disclosure will be described. In the embodiments, as an example of an endoscope apparatus according to the present disclosure, a medical endoscope apparatus for capturing an image of the inside of an object to be examined, such as a patient, and displaying the image will be described. In addition, this disclosure is not limited by the embodiments. In addition, in the description of the diagrams, the same reference numerals are given to the same units.

### First Embodiment

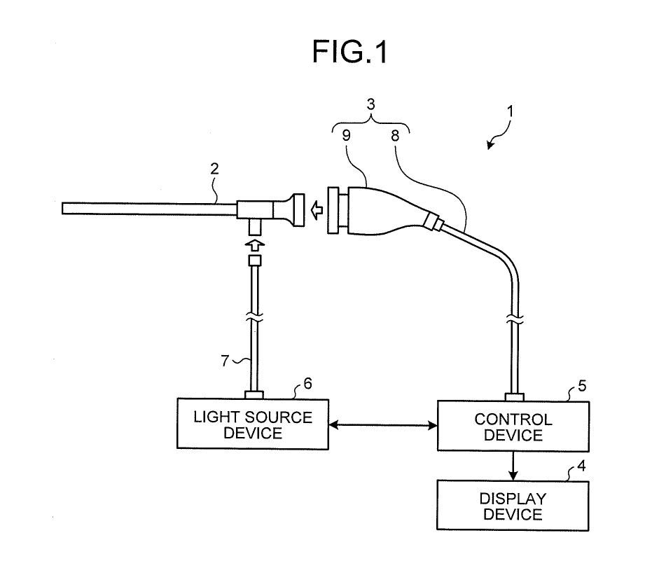

FIG. 1 is a diagram illustrating the schematic configuration of an endoscope apparatus 1 according to a first embodiment of the present disclosure. The endoscope apparatus 1 is an apparatus that is used in a medical field in order to observe a subject inside an observation target (inside a living body), such as a person. As illustrated in FIG. 1, the endoscope apparatus 1 includes an endoscope 2, an imaging device 3, a display device 4, a control device 5, and a light source device 6.

The light source device 6, to which one end of a light guide 7 is connected, has a light source unit 61 that supplies illumination light, for example, white light for illuminating the inside of the living body or infrared light for infrared observation, to the one end of the light guide 7, and a light source controller 62 that controls emission of illumination light from the light source unit 61. As a light source provided in the light source unit 61, for example, a halogen lamp, a xenon lamp, a Light Emitting Diode (LED), or a Laser Diode (LD) is used.

One end of the light guide 7 is detachably connected to the light source device 6, and the other end of the light guide 7 is detachably connected to the endoscope 2. Then, the light guide 7 transmits light supplied from the light source device 6 from the one end through the other end to supply the light to the endoscope 2.

The imaging device 3 captures a subject image from the endoscope 2 and outputs the captured subject image. As illustrated in FIG. 1, the imaging device 3 includes a transmission cable 8, which is a signal transmission unit, and a camera head 9. In the first embodiment, a medical imaging device is configured by the transmission cable 8 and the camera head 9.

The endoscope 2 is rigid and of an elongated shape. The endoscope 2 is inserted into a living body. The endoscope 2 is provided with an observation optical system configured of one or a plurality of lenses that condenses a subject image. In addition, the endoscope 2 is provided at the distal end thereof with a cover glass. The endoscope 2 emits light, which is supplied through the light guide 7, from the distal end to the inside of the living body. Then, the light (subject image) emitted to and then reflected by the inside of the living body is guided by the observation optical system (endoscope side optical system 21A) in the endoscope 2.

The camera head 9 is detachably connected to the proximal end of the endoscope 2. Under the control of the control device 5, the camera head 9 captures the subject image condensed by the endoscope 2 and outputs an imaging signal obtained by capturing the subject image. The detailed configuration of the camera head 9 will be described later. The endoscope 2 and the camera head 9 may be detachably configured as illustrated in FIG. 1 or may be integrated.

One end of the transmission cable 8 is detachably connected to the control device 5 through a connector, and the other end of the transmission cable 8 is detachably connected to the camera head 9 through a connector.

Specifically, the transmission cable 8 is a cable in which a plurality of electric wires (not illustrated) are disposed inside of an outer coat serving as the outermost layer. The plurality of electric wires are electric wires for transmitting the imaging signal from the camera head 9 to the control device 5 and transmitting a control signal, a synchronization signal, a clock signal, and electric power, which are output from the control device 5, to the camera head 9.

Under the control of the control device 5, the display device 4 displays an image generated by the control device 5. In order for a user to concentrate on the observation of the subject, the display device 4 preferably has a display unit of 55 inch size or larger, but the display device 4 is not limited thereto.

The control device 5 processes the imaging signal input from the camera head 9 through the transmission cable 8 and outputs an image signal to the display device 4. The control device 5 comprehensively controls the operations of the camera head 9 and the display device 4. The detailed configuration of the control device 5 will be described later.

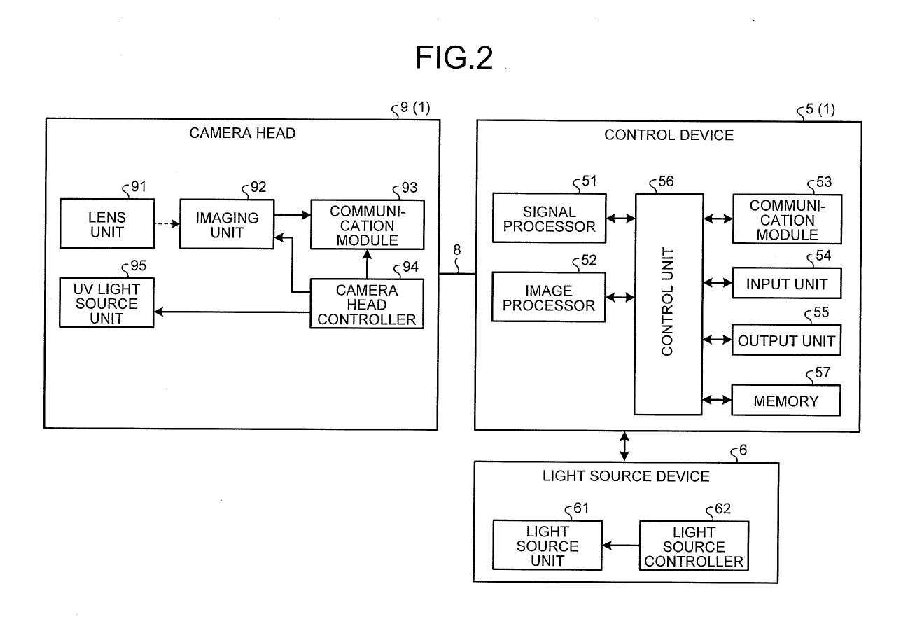

Next, the configuration of the imaging device 3 and the control device 5 will be described. FIG. 2 is a block diagram illustrating the configuration of the camera head 9 and the control device 5. In FIG. 2, a connector is omitted which allows the camera head 9 and the transmission cable 8 to be attachable to and detachable from each other.

Hereinafter, the configurations of the control device 5 and the configuration of the camera head 9 will be described in this order. The following description is focused on main units of the control device 5. As illustrated in FIG. 2, the control device 5 includes a signal processor 51, an image processor 52, a communication module 53, an input unit 54, an output unit 55, a control unit 56, and a memory 57. In addition, the control device 5 may be provided with a power supply unit (not illustrated) that generates a power supply voltage for driving the control device 5 and the camera head 9 and supplies the power supply voltage to each unit of the control device 5 and to the camera head 9 through the transmission cable 8 and the like.

The signal processor 51 performs signal processing, such as noise removal or A/D conversion as necessary, on the imaging signal output from the camera head 9 and outputs a digitized imaging signal (pulse signal) to the image processor 52.

In addition, the signal processor 51 generates a synchronization signal and a clock signal for the imaging device 3 and the control device 5. The synchronization signal (for example, a synchronization signal indicating the imaging timing of the camera head 9) or the clock signal (for example, a clock signal for serial communication) to the imaging device 3 is transmitted to the imaging device 3 through a line (not illustrated), and the imaging device 3 is driven based on the synchronization signal or the clock signal.

Based on the imaging signal input from the signal processor 51, the image processor 52 generates a display image signal to be displayed by the display device 4. The image processor 52 generates a display image signal including a subject image by performing predetermined signal processing on the imaging signal. Here, the image processor 52 performs known image processing including various kinds of image processing, such as detection processing, interpolation processing, color correction processing, color enhancement processing, and edge enhancement processing. The image processor 52 outputs the generated image signal to the display device 4.

The communication module 53 outputs a signal from the control device 5, which includes a control signal (to be described later) transmitted from the control unit 56, to the imaging device 3. In addition, a signal from the imaging device 3 is output to each unit of the control device 5. That is, the communication module 53 is a relay device that performs, for example, a parallel-serial conversion on the signals from the respective units of the control device and collectively outputs a converted signal to the imaging device 3, and performs, for example, a serial-parallel conversion the signal input from the imaging device 3 and parallelly outputs converted signals to the respective units of the control device 5.

The input unit 54 is realized by using a user interface, such as a keyboard, a computer mouse, and a touch panel, and receives an input of various kinds of information.

The output unit 55 is realized by using a speaker, a printer, a display, or the like, and outputs various kinds of information.

The control unit 56 performs driving control with respect to respective components including the control device 5 and the camera head 9, input and output control of information with respect to the respective components, and the like. The control unit 56 generates a control signal by referring to communication information data (for example, communication format information) recorded in the memory 57, and transmits the generated control signal to the imaging device 3 through the communication module 53. In addition, the control unit 56 outputs a control signal to the camera head 9 through the transmission cable 8. The control unit 56 switches the wavelength band of illumination light emitted from the light source device 6 according to an observation method switching instruction input through the input unit 54, for example. As observation methods, there are normal observation in which white light is emitted and special light observation in which light in a wavelength band different from the white wavelength band is emitted. In the first embodiment, as an example, infrared observation in which light in an infrared wavelength band is emitted to the subject in order to observe the subject under infrared light is referred to as special light observation.

The memory 57 is realized by using a semiconductor memory, such as a flash memory or a Dynamic Random Access Memory (DRAM), and records communication information data (for example, communication format information).

Incidentally, various programs executed by the control unit 56 and the like may be recorded in the memory 57.

Incidentally, the signal processor 51 may have an AF processor that outputs a predetermined AF evaluation value of each frame based on the imaging signal of the input frame and an AF calculation unit that performs AF calculation processing for selecting a frame, a focus lens position, or the like, which is most suitable as a focus position, from the AF evaluation value of each frame from the AF processor.

The signal processor 51, the image processor 52, the communication module 53, and the control unit 56 described above are realized by general-purpose processors, such as a Central Processing Unit (CPU) having an internal memory (not illustrated) in which a program is recorded, or dedicated processors, such as various calculation circuits for executing specific functions including an Application Specific Integrated Circuit (ASIC). Alternatively, the signal processor 51, the image processor 52, the communication module 53, and the control unit 56 may be configured using a Field Programmable Gate Array (FPGA: not illustrated) that is one type of programmable integrated circuit. Incidentally, in a case where the signal processor 51, the image processor 52, the communication module 53, and the control unit 56 are configured by the FPGA, a memory that stores configuration data may be provided, and the FPGA, which is a programmable integrated circuit, may be configured by the configuration data read from the memory.

Next, the configuration of the camera head 9 will mainly be described. As illustrated in FIG. 2, the camera head 9 includes a lens unit 91 that is a part of the observation optical system, an imaging unit 92, a communication module 93, and a camera head controller 94. Incidentally, in the first embodiment, as will be described later, the camera head 9 may take a configuration including an observation side filter that cuts light in a predetermined wavelength band or a configuration not including the filter.

The lens unit 91 is configured using one or a plurality of lenses, and forms an incident subject image on the imaging surface of an imaging element that configures the imaging unit 92. The one or a plurality of lenses are configured to be movable along the optical axis. Additionally, the lens unit 91 is provided with an optical zoom mechanism (not illustrated) for changing the angle of view by moving the one or a plurality of lenses and a focus mechanism for changing the focal position by moving the one or a plurality of lenses. Incidentally, the lens unit 91 forms an observation optical system for guiding observation light, which has entered to the endoscope 2, to the imaging unit 92 together with an optical system provided in the endoscope 2.

Under the control of the camera head controller 94, the imaging unit 92 captures a subject image. The imaging unit 92 is configured using an imaging element that receives a subject image formed by the lens unit 91 and converts the subject image into an electric signal. The imaging element is configured by a Charge Coupled Device (CCD) image sensor or a Complementary Metal Oxide Semiconductor (CMOS) image sensor. In a case where the imaging element is a CCD, for example, a signal processor (not illustrated) that performs signal processing (A/D conversion or the like) on the electric signal (analog signal) from the imaging element and outputs an imaging signal is mounted on a sensor chip or the like. In a case where the imaging element is a CMOS, for example, a signal processor (not illustrated) that performs signal processing (A/D conversion or the like) on an electric signal (analog signal) converted from light and outputs an imaging signal is included in the imaging element. The imaging unit 92 outputs the generated electric signal to the communication module 93.

The communication module 93 outputs a signal transmitted from the control device 5 to each unit in the camera head 9, such as the camera head controller 94. In addition, the communication module 93 converts information regarding the current state of the camera head 9 or the like in a signal format corresponding to a predetermined transmission method, and outputs the converted signal to the control device 5 through the transmission cable 8. That is, the communication module 93 is a relay device that performs, for example, a serial-parallel conversion on a signal input from the control device 5 through the transmission cable 8 and parallelly outputs converted signals to the respective units of the camera head 9, and performs, for example, a parallel-serial conversion on signals from the respective units of the camera head 9 and collectively outputs a converted signal to the control device 5 through the transmission cable 8.

The camera head controller 94 controls the operation of the entire camera head 9 according to a driving signal input through the transmission cable 8, an instruction signal that is output from an operating unit, such as a switch provided on the outer surface of the camera head 9 so as to be exposed, by the user's operation on the operating unit, and the like. In addition, the camera head controller 94 outputs the information regarding the current state of the camera head 9 to the control device 5 through the transmission cable 8.

The communication module 93 and the camera head controller 94 described above are realized by using general-purpose processors, such as a CPU having an internal memory (not illustrated) in which a program is recorded, or dedicated processors, such as various calculation circuits for executing specific functions including an ASIC. Alternatively, the communication module 93 and the camera head controller 94 may be configured using an FPGA, which is one type of programmable integrated circuit. Here, in a case where the communication module 93 and the camera head controller 94 are configured by the FPGA, a memory that stores configuration data may be provided, and the FPGA, which is a programmable integrated circuit, may be configured by the configuration data read from the memory.

In addition, a signal processor that performs signal processing on the imaging signal generated by the communication module 93 or the imaging unit 92 may be provided in the camera head 9 or the transmission cable 8. In addition, an imaging clock for driving the imaging unit 92 and a control clock signal for the camera head controller 94 may be generated based on a reference clock signal generated by an oscillator (not illustrated) provided in the camera head 9 and the imaging clock signal and the control clock signal may be output to the imaging unit 92 and the camera head controller 94, respectively, or timing signals for various kinds of processing in the imaging unit 92 and the camera head controller 94 may be generated based on a synchronization signal input from the control device 5 through the transmission cable 8 and the timing signals may be output to the imaging unit 92 and the camera head controller 94, respectively. Alternatively, the camera head controller 94 may be provided in the transmission cable 8 or the control device 5 instead of the camera head 9.

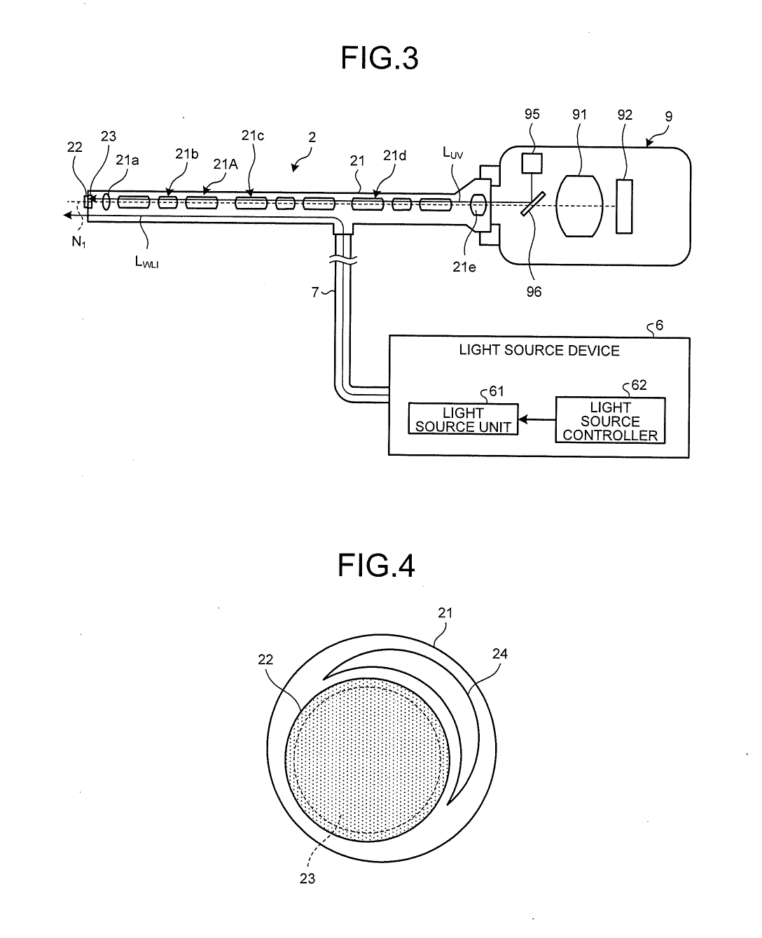

FIG. 3 is a schematic diagram illustrating the configuration of the endoscope 2 and the camera head 9 according to the embodiment of the present disclosure. FIG. 4 is a schematic diagram illustrating the distal end configuration of the endoscope according to the first embodiment of the present disclosure, and is a plan view illustrating the configuration of the distal end surface of the endoscope 2. The endoscope 2 takes in external light at the distal end thereof and is electrically connected at the proximal end thereof to the camera head 9.

The endoscope 2 includes the endoscope side optical system 21A, which is a part of the observation optical system, inside an insertion unit 21 (for example, refer to FIG. 3). The endoscope side optical system 21A includes an objective lens 21a, a first relay optical system 21b, a second relay optical system 21c, a third relay optical system 21d, and an eyepiece 21e in this order from the distal end side along an optical axis N₁ of the endoscope side optical system 21A.

An ultraviolet light absorption filter 22 (hereinafter, referred to as a UV absorption filter 22), a cover glass 23, and an illumination window 24 (FIG. 4) are provided at the distal end of the endoscope 2. The cover glass 23 is provided at the distal end of the endoscope side optical system 21A on the observation light incident side. The cover glass 23 is an incident window on which observation light from the subject is incident, and serves as an incident end portion in the endoscope 2. The UV absorption filter 22 covers a surface of the cover glass 23 on a side opposite to the side of the endoscope side optical system 21A (refer to FIG. 4). That is, the UV absorption filter 22 is provided at the incident end portion in the distal end of the endoscope 2, and covers the incident end portion. The UV absorption filter 22 is, for example, a filter that absorbs light (ultraviolet light) in a wavelength band of 400 nm or less and generates heat. The UV absorption filter 22 may be provided on the cover glass 23 by coating, or may be attached to the cover glass 23 with an adhesive sheet such as a seal. In addition, the illumination window 24 is a window through which illumination light for illuminating the subject is emitted from the endoscope 2, and serves as an emission end portion in the endoscope 2.

In the camera head 9, the lens unit 91 and the imaging unit 92 are disposed in this order from one end to which the endoscope 2 is connected. The optical axes of the lens unit 91 and the imaging unit 92 align with the optical axis N₁ of an endoscope side optical system 21A. In this specification, the observation optical system for guiding the observation light to the imaging unit 92 is formed by the endoscope side optical system 21A and the lens unit 91.

In addition, the camera head 9 is provided with a UV light source unit 95 that emits ultraviolet light and a dichroic mirror 96 disposed on the optical axis of the lens unit 91 are provided. The UV light source unit 95 emits ultraviolet light under the control of the camera head controller 94. The UV light source unit 95 is configured using an LED that emits ultraviolet light. The dichroic mirror 96 reflects ultraviolet light in a direction in parallel to the optical path of the observation light and toward the endoscope side optical system 21A, and transmits light in a wavelength band other than the ultraviolet wavelength band. Ultraviolet light L_{UV} emitted from the UV light source unit 95 is reflected by the dichroic mirror 96, and then travels along the optical axis N₁ to the UV absorption filter 22.

On the other hand, white light or infrared light is supplied from the light source device 6 to the endoscope 2 through the light guide 7, and is emitted to the outside from the illumination window 24 (for example, white light L_{WLI} illustrated in FIG. 3). At the time of normal observation, all the light beams configuring the white light enter the imaging unit 92. The white light includes light in a blue wavelength band, light in a green wavelength band, and light in a red wavelength band. The red wavelength band includes an infrared wavelength band.

However, the red wavelength band may not include the infrared wavelength band. On the other hand, at the time of special light observation (infrared observation), for example, infrared light is emitted from the light source device 6, and the imaging unit 92 receives the infrared light from an observed region. Incidentally, excitation light that is not infrared light may be emitted from the light source device 6, and the imaging unit 92 may receive infrared light that is fluorescence from the subject due to the excitation light.

At the time of normal observation and infrared observation, ultraviolet light is emitted from the UV light source unit 95. As a result, the UV absorption filter 22 is irradiated with ultraviolet light. The UV absorption filter 22 generates heat by absorbing the ultraviolet light. By the heat generation of the UV absorption filter 22, the temperature difference between the temperature of the body cavity and the temperature of the UV absorption filter 22 or the cover glass 23 is reduced. As a result, the occurrence of dew condensation is suppressed. Incidentally, the UV light source unit 95 is controlled to emit light whose amount is saturated when the temperature of the UV absorption filter 22 is 37° C. or higher and 41° C. or lower.

In the first embodiment described above, the surface of the cover glass 23 serving as an entrance, through which light enters the observation optical system 21A from the observed region, at the distal end of the endoscope 2 is covered with the UV absorption filter 22, and ultraviolet light from the UV light source unit 95 of the camera head 9 is emitted to the UV absorption filter 22. In the first embodiment, the UV absorption filter 22 provided at the distal end of the endoscope 2 can be heated to suppress the occurrence of dew condensation, and light in the infrared wavelength band can pass through the dichroic mirror 96 and the imaging unit 92 can receive the light. According to the first embodiment, since the UV absorption filter 22 may be provided at the distal end of the endoscope 2, it is possible to suppress an increase in the diameter of the insertion unit 21.

In addition, according to the first embodiment described above, since the dichroic mirror 96 is provided to separate ultraviolet light and light in other wavelength bands, it is not necessary to provide a dedicated light guiding unit that guides ultraviolet light to the UV absorption filter 22. Therefore, an increase in the diameter of the insertion unit 21 can be suppressed, and the ultraviolet light can be efficiently guided to the UV absorption filter 22.

Incidentally, in the first embodiment described above, the UV absorption filter 22 and the cover glass 23 may be integrated. That is, the cover glass 23 may be formed of a material that absorbs ultraviolet light. In this case, the cover glass 23 configures an incident end portion of the endoscope 2, and functions as a UV absorption filter.

In addition, in the first embodiment described above, in the case of performing only normal light observation, a dichroic mirror configured to transmit at least light having a wavelength in a visible region may be used as the dichroic mirror 96. In addition, as the dichroic mirror 96, a dichroic mirror in which the wavelength band of reflected light and the light of transmitted wavelength band are switched around may be used depending on the arrangement of the imaging unit 92 and the UV light source unit 95. Specifically, in a case where the wavelength band of reflected light and the light of transmitted wavelength band are reversed depending on the arrangement of the imaging unit 92 and the UV light source unit 95, the dichroic mirror 96 transmits ultraviolet light and reflects light in a wavelength band other than the ultraviolet wavelength band.

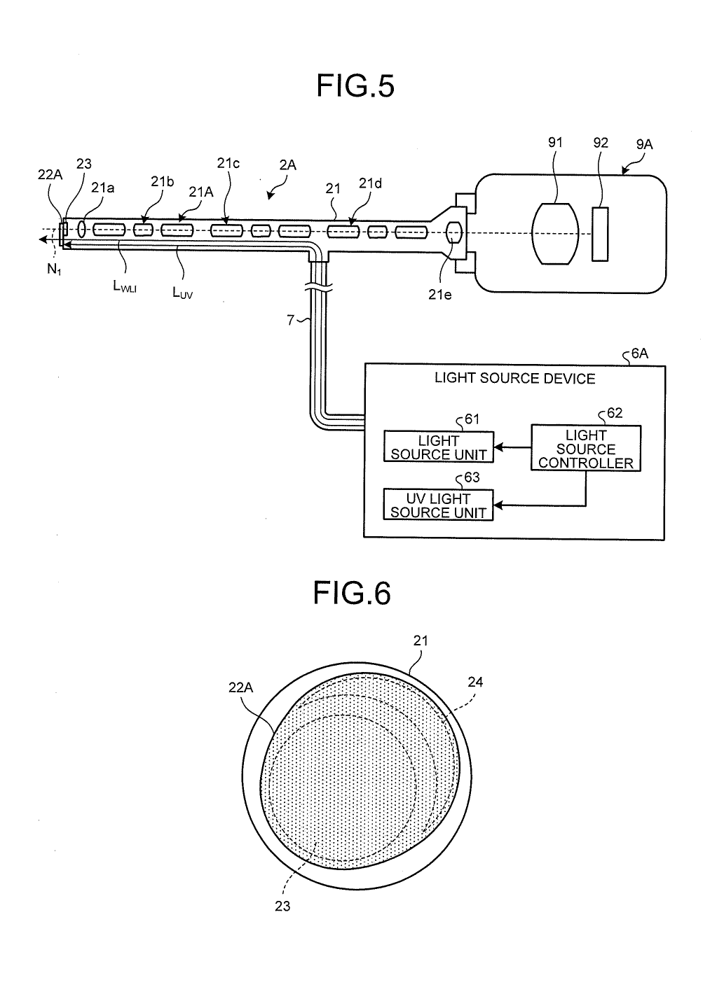

First modification of the first embodiment Next, a first modification of the first embodiment of the present disclosure will be described. FIG. 5 is a schematic diagram illustrating the configuration of an endoscope and a light source device according to the first modification of the first embodiment of the present disclosure. FIG. 6 is a schematic diagram illustrating the distal end configuration of the endoscope according to the first modification of the first embodiment of the present disclosure, and is a plan view illustrating the configuration of the distal end surface of an endoscope 2A. An endoscope apparatus according to the present first modification is different from the endoscope apparatus 1 described above only in the arrangement of a UV light source unit that emits ultraviolet light and the covering range of a UV absorption filter. The other configuration is the same as that of the endoscope apparatus 1 described above. Hereinafter, portions different from the above first embodiment will be described.

The endoscope apparatus according to the present first modification includes the endoscope 2A, an imaging device (a transmission cable 8 and a camera head 9A), a display device 4, a control device 5, and a light source device 6A.

The light source device 6A, to which one end of a light guide 7 is connected, has a light source unit 61 that supplies illumination light, for example, white light for illuminating the inside of the living body or infrared light for infrared observation, to the one end of the light guide 7, a light source controller 62 that controls emission of illumination light from the light source unit 61, and a UV light source unit 63 that emits ultraviolet light.

The endoscope 2A is rigid and of elongated shape. The endoscope 2A is inserted into a living body. The endoscope 2A has the endoscope side optical system 21A described above, a UV absorption filter 22A, and a cover glass 23. The UV absorption filter 22A is a filter that absorbs light (ultraviolet light) in a wavelength band of 400 nm or less and generates heat. The UV absorption filter 22A covers the cover glass 23 and the illumination window 24 (refer to FIG. 6). In other words, the UV absorption filter 22A is attached to both the cover glass 23, which is an incident end portion, and the illumination window 24, which is an emission end portion.

The camera head 9A has the lens unit 91, the imaging unit 92, the communication module 93 (FIG. 2), and the camera head controller 94 (FIG. 2) described above. The camera head 9A is not provided with the UV light source unit 95 and the dichroic mirror 96, differently from the camera head 9 described above.

White light or infrared light supplied from the light source unit 61 is guided to the endoscope 2 through the light guide 7 and is emitted to the outside from the illumination window 24 (for example, white light L_{WLI} illustrated in FIG. 5). In addition, ultraviolet light L_{UV} emitted from the UV light source unit 63 travels along the illumination optical path after having passed through the light guide 7 to be emitted to the UV absorption filter 22A.

At the time of normal observation and infrared observation, in addition to white light or infrared light, ultraviolet light is emitted from the UV light source unit 63. As a result, the UV absorption filter 22A is irradiated with ultraviolet light. The UV absorption filter 22A generates heat by absorbing the ultraviolet light. The UV absorption filter 22A is heated from the side of the illumination window 24, and the heat is transmitted to the side of the cover glass 23. By the heat generation of the UV absorption filter 22A, the temperature difference between the temperature of the body cavity and the temperature of the UV absorption filter 22A or the cover glass 23 is reduced. As a result, the occurrence of dew condensation is suppressed.

According to the first modification described above, as in the first embodiment, it is possible to suppress dew condensation at the distal end of the insertion unit 21 while suppressing an increase in the diameter of the insertion unit 21 and to perform observation using infrared light.

### Second Modification of the First Embodiment

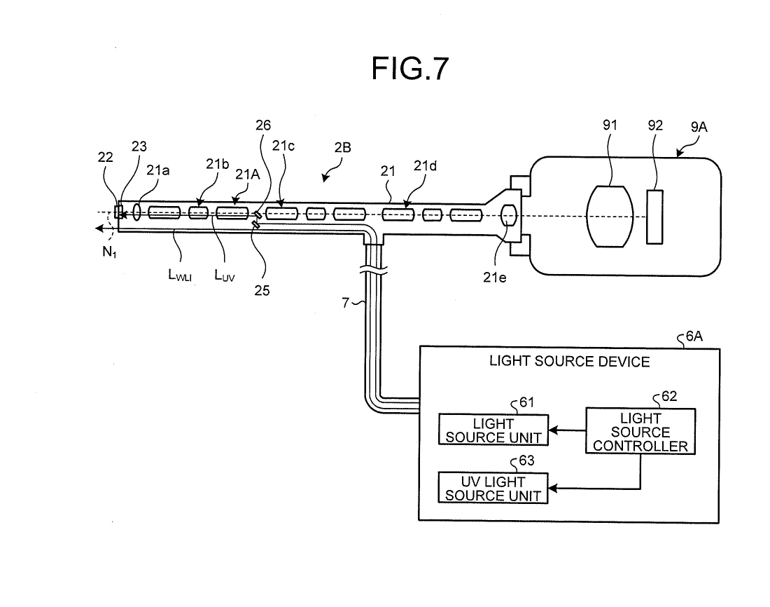

Next, a second modification of the first embodiment of the present disclosure will be described. FIG. 7 is a schematic diagram illustrating the configuration of an endoscope and a light source device according to the second modification of the first embodiment of the present disclosure. An endoscope apparatus according to the present second modification is different from the endoscope apparatus 1 described above only in the arrangement of a UV light source unit that emits ultraviolet light and a configuration for making ultraviolet light enter the observation optical system. The other configuration is the same as that of the endoscope apparatus 1 described above. Hereinafter, portions different from the above first embodiment will be described.

The endoscope apparatus according to the present second modification includes an endoscope 2B, an imaging device (a transmission cable 8 and a camera head 9A), a display device 4, a control device 5, and a light source device 6A.

The light source device 6A has the same configuration as that of the first modification described above. White light or infrared light supplied from the light source unit 61 is guided to the endoscope 2 through the light guide 7 and is emitted to the outside from the illumination window (for example, white light L_{WLI} illustrated in FIG. 7).

The endoscope 2B is rigid and of elongated shape. The endoscope 2B is inserted into a living body. The endoscope 2B has the endoscope side optical system 21A, the UV absorption filter 22, and the cover glass 23 described above, a first dichroic mirror 25, and a second dichroic mirror 26. The first dichroic mirror 25 is provided on the optical path of illumination light, and reflects ultraviolet light and transmits light in a wavelength band other than the ultraviolet wavelength band. The second dichroic mirror 26 is provided on the optical axis N₁ of the observation optical system, and reflects ultraviolet light and transmits light in a wavelength band other than the ultraviolet wavelength band. The ultraviolet light L_{UV} emitted from the UV light source unit 63 travels along the illumination optical path after having passed through the light guide 7 and is reflected by the first dichroic mirror 25 and the second dichroic mirror 26, and travels farther along the optical path of the observation optical system to be emitted to the UV absorption filter 22.

The camera head 9A has the lens unit 91, the imaging unit 92, the communication module 93, and the camera head controller 94 described above. The camera head 9A has the same configuration as that of the first modification described above.

At the time of normal observation and infrared observation, in addition to white light or infrared light, ultraviolet light is emitted from the UV light source unit 63. As a result, the UV absorption filter 22 is irradiated with ultraviolet light. The UV absorption filter 22 generates heat by absorbing the ultraviolet light. By the heat generation of the UV absorption filter 22, the temperature difference between the temperature of the body cavity and the temperature of the UV absorption filter 22 or the cover glass 23 is reduced. As a result, the occurrence of dew condensation is suppressed.

According to the second modification described above, as in the first embodiment, it is possible to suppress dew condensation at the distal end of the insertion unit 21 while suppressing an increase in the diameter of the insertion unit 21 and to perform observation using infrared light.

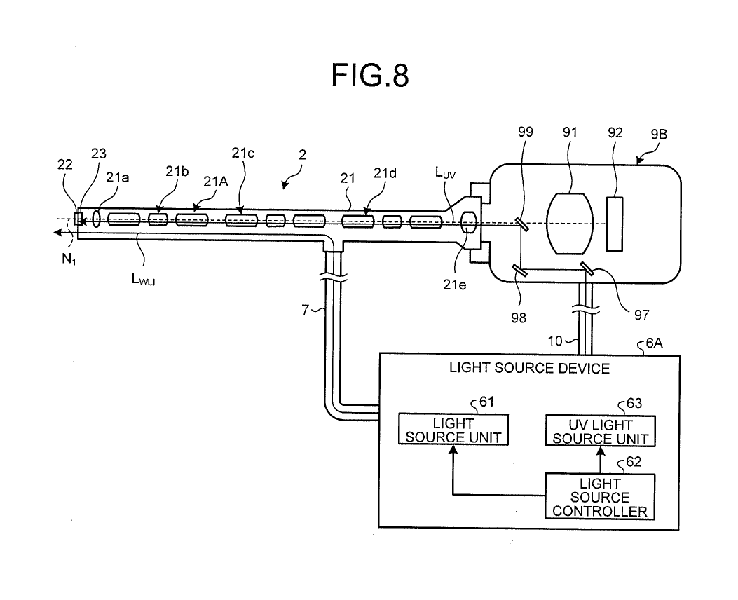

Third modification of the first embodiment Next, a third modification of the first embodiment of the present disclosure will be described. FIG. 8 is a schematic diagram illustrating the configuration of an endoscope and a light source device according to the third modification of the first embodiment of the present disclosure. An endoscope apparatus according to the present third modification is different from the endoscope apparatus 1 described above only in the arrangement of a UV light source unit that emits ultraviolet light and a configuration for making ultraviolet light enter the observation optical system. The other configuration is the same as that of the endoscope apparatus 1 described above. Hereinafter, portions different from the above first embodiment will be described.

The endoscope apparatus according to the present third modification includes an endoscope 2, an imaging device (a transmission cable 8 and a camera head 9B), a display device 4, a control device 5, and a light source device 6A. In the third modification, a light guide 10 that connects the light source device 6A and the camera head 9B to each other is further provided.

The light source device 6A has the same configuration as that of the first modification described above. White light or infrared light supplied from the light source unit 61 is guided to the endoscope 2 through the light guide 7 and is emitted to the outside from the illumination window (for example, white light L_{WLI} illustrated in FIG. 8).

The camera head 9B has the lens unit 91, the imaging unit 92, the communication module 93, and the camera head controller 94 described above, a first deflection mirror 97, a second deflection mirror 98, and a dichroic mirror 99. The dichroic mirror 99 reflects ultraviolet light and transmits light in a wavelength band other than the ultraviolet wavelength band.

The ultraviolet light L_{UV} emitted from the UV light source unit 63 is guided to the camera head 9B through the light guide 10. The ultraviolet light L_{UV}, which has entered to the camera head 9B, enters the optical path of the endoscope side optical system 21A through the first deflection mirror 97, the second deflection mirror 98, and the dichroic mirror 99. The ultraviolet light L_{UV} that has entered the optical path of the endoscope side optical system 21A travels along the optical axis N₁ to be emitted to the UV absorption filter 22.

At the time of normal observation and infrared observation, in addition to white light or infrared light, the ultraviolet light is emitted from the UV light source unit 63. As a result, the UV absorption filter 22 is irradiated with ultraviolet light. By the heat generation of the UV absorption filter 22 using the ultraviolet light, the temperature difference between the temperature of the body cavity and the temperature of the UV absorption filter 22 or the cover glass 23 is reduced. As a result, the occurrence of dew condensation is suppressed.

According to the third modification described above, as in the first embodiment, it is possible to suppress dew condensation at the distal end of the insertion unit 21 while suppressing an increase in the diameter of the insertion unit 21 and to perform observation using infrared light.

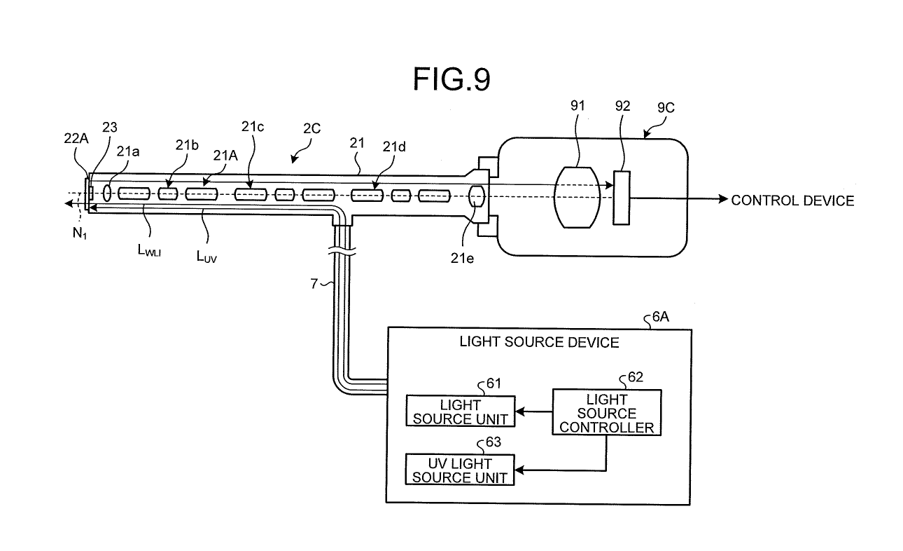

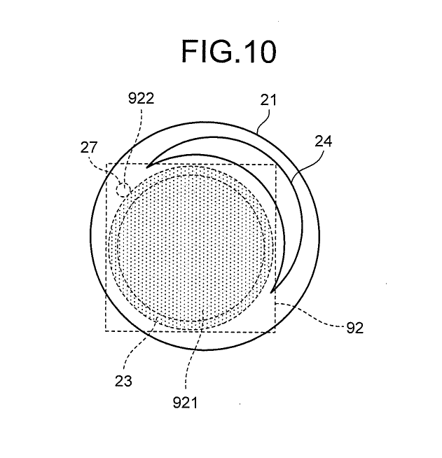

Fourth modification of the first embodiment Next, a fourth modification of the first embodiment of the present disclosure will be described. FIG. 9 is a schematic diagram illustrating the configuration of an endoscope and a light source device according to the fourth modification of the first embodiment of the present disclosure. FIG. 10 is a schematic diagram illustrating the distal end configuration of the endoscope according to the fourth modification of the first embodiment of the present disclosure, and is a plan view illustrating the configuration of the distal end surface of an endoscope 2B. An endoscope apparatus according to the present fourth modification further includes a configuration for measuring the temperature of the UV absorption filter 22 in the configuration of the first modification described above. Hereinafter, portions different from the above first modification will be described.

The endoscope apparatus according to the present fourth modification includes an endoscope 2C, an imaging device (a transmission cable 8 and a camera head 9C), a display device 4, a control device 5, and a light source device 6A.

The light source device 6A has the same configuration as that of the first modification described above. White light or infrared light supplied from the light source unit 61 is guided to the endoscope 2 through the light guide 7 and is emitted to the outside from the illumination window (for example, white light L_{WLI} illustrated in FIG. 9). In addition, ultraviolet light L_{UV} emitted from the UV light source unit 63 travels along the illumination optical path after having passed through the light guide 7 to be emitted to the UV absorption filter 22A.

The endoscope 2C is rigid and of elongated shape. The endoscope 2C is inserted into a living body. The endoscope 2C has the endoscope side optical system 21A described above, the UV absorption filter 22A, and the cover glass 23, an illumination window 24 (FIG. 10), and a temperature measurement window 27 (FIG. 10).

The camera head 9C has the lens unit 91, the imaging unit 92, the communication module 93 (FIG. 2), and the camera head controller 94 (FIG. 2) described above. The camera head 9C has the same configuration as that of the camera head 9A of the first modification described above.

At the time of normal observation and infrared observation, in addition to white light or infrared light, ultraviolet light is emitted from the UV light source unit 63. As a result, the UV absorption filter 22A is irradiated with the ultraviolet light. By the heat generation of the UV absorption filter 22A due to absorption of ultraviolet light, the temperature difference between the temperature of the body cavity and the temperature of the UV absorption filter 22 or the cover glass 23 is reduced. As a result, the occurrence of dew condensation is suppressed.

At this time, when the UV absorption filter 22A generates heat, infrared light is emitted from the surface of the UV absorption filter 22A. In the fourth modification, the temperature of the UV absorption filter 22A is measured by measuring the infrared light emitted from the surface of the UV absorption filter 22A. The infrared light emitted from the surface of the UV absorption filter 22A is incident on the imaging unit 92 through the insertion unit 21. In this case, the infrared light may be guided using a light guide, or may be guided along the optical path formed by the optical system. An infrared light receiving unit 922 (FIG. 10) in the imaging unit 92 is a region different from a light receiving region 921 of observation light guided by the endoscope side optical system 21A. The imaging unit 92 photoelectrically converts the received infrared light and outputs the generated electric signal to the control device 5. In the control device 5, the control unit 56 (FIG. 2) measures the temperature of the UV absorption filter 22A from the signal value of the electric signal. In this manner, by separating the light receiving region of observation light from the light receiving region of infrared light due to heat generation and performing signal processing separately, it is possible to independently measure the temperature of the UV absorption filter 22A. According to the measurement result, the control unit 56 controls the intensity (including zero) of ultraviolet light from the UV light source unit 63, for example, performs control such that the temperature of the UV absorption filter 22A is 37° C. or higher and 41° C. or lower.

According to the fourth modification described above, as in the first embodiment, it is possible to suppress dew condensation at the distal end of the insertion unit 21 while suppressing an increase in the diameter of the insertion unit 21 and to perform observation using infrared light. In addition, according to the present fourth modification, the temperature of the UV absorption filter 22 can be appropriately controlled to be a set temperature.

### Second Embodiment

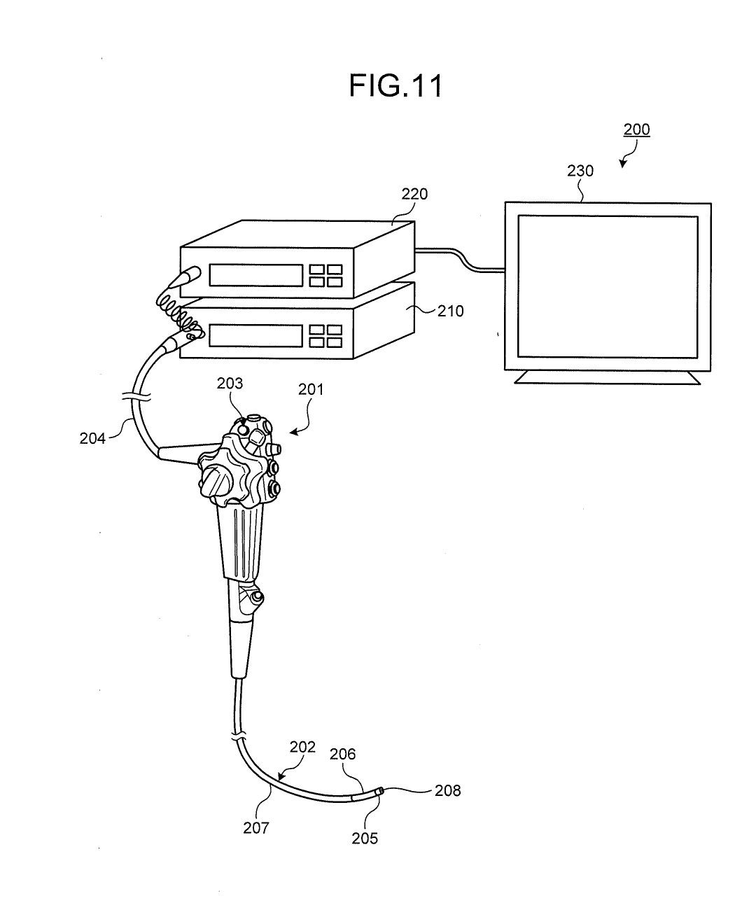

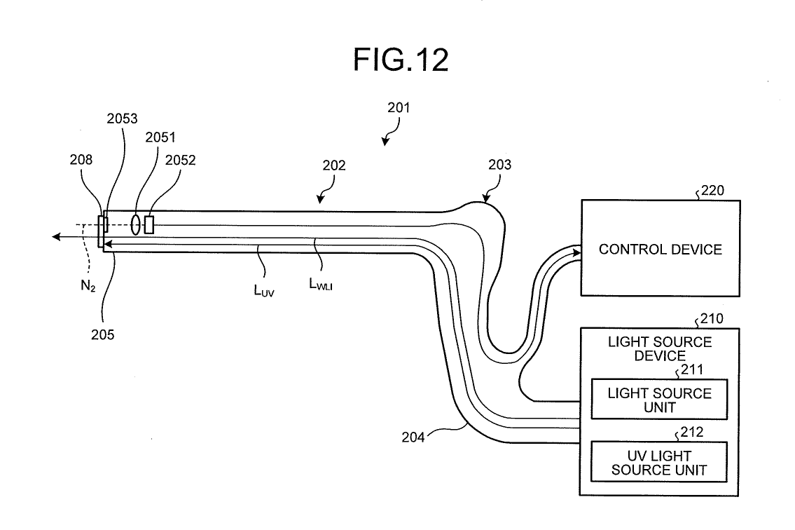

Next, a second embodiment of the present disclosure will be described. FIG. 11 is a diagram illustrating the schematic configuration of an endoscope apparatus 200 according to a second embodiment of the present disclosure. FIG. 12 is a schematic diagram illustrating the configuration of an endoscope 201 and a light source device 210 according to the second embodiment of the present disclosure. In the first embodiment described above, the endoscope apparatus 1 using a rigid endoscope as the endoscope 2 has been described. However, the present disclosure is not limited thereto, and an endoscope apparatus using a flexible endoscope may be used. In the present second embodiment, an example in a case where a UV absorption filter is provided at the distal end of an insertion unit of a flexible endoscope will be described.

The endoscope apparatus 200 includes the endoscope 201 that captures an in-vivo image of an observed region by an insertion unit 202 inserted into an object to be examined, and generates an imaging signal, the light source device 210 that generates illumination light to be emitted from the distal end of the endoscope 201, a control device 220 that performs predetermined image processing on the imaging signal acquired by the endoscope 201 and performs overall control of the operation of the entire endoscope apparatus 200, and a display device 230 that displays an in-vivo image subjected to the image processing by the control device 220. The endoscope apparatus 200 acquires an in-vivo image of the inside of an object to be examined, such as a patient, by inserting the insertion unit 202 into the object. In addition, the control device 220 has the functions of the signal processor 51, the image processor (FIG. 2), and the like described above.

The endoscope 201 includes the insertion unit 202 that has flexibility and has an elongated shape, an operating unit 203 that is connected to the proximal end side of the insertion unit 202 and receives an input of various operation signals, and a universal cord 204 that extends from the operating unit 203 in a direction different from the extension direction of the insertion unit 202 and contains thereinside various cables connected to the light source device 210 and the control device 220.

The insertion unit 202 has a distal end portion 205 that contains an imaging unit thereinside, a bending portion 206 that is configured by a plurality of bending pieces and can be bent, and a flexible tube portion 207 that is connected to the proximal end side of the bending portion 206, has flexibility, and has an elongated shape.

Referring to FIG. 12, the distal end portion 205 includes a lens unit 2051, an imaging unit 2052, and a cover glass 2053. The lens unit 2051 is configured using one or a plurality of lenses, and forms a subject image incident through the cover glass 2053 on the imaging surface of an imaging element that configures the imaging unit 2052. Under the control of the control device 220, the imaging unit 2052 captures a subject image. The imaging unit 2052 is configured using an imaging element that receives the subject image formed by the lens unit 2051 and converts the subject image into an electric signal. The imaging element is configured by a Charge Coupled Device (CCD) image sensor or a Complementary Metal Oxide Semiconductor (CMOS) image sensor. The lens unit 2051 and the imaging unit 2052 are arranged along an optical axis N₂. The imaging unit 2052 outputs the generated electric signal to the control device 220 through the insertion unit 202 and the operating unit 203.

In addition, the outer surface of the cover glass 2053 of the distal end portion 205 and the outer surface of an illumination window (not illustrated), through which illumination light from a light source unit 211 is emitted, are covered with an UV absorption filter 208 (FIG. 12). The UV absorption filter 208 is a filter that absorbs light (ultraviolet light) in a wavelength band of 400 nm or less.

The light source device 210 includes the light source unit 211 capable of performing switching between the emission of white light and the emission of infrared light and a UV light source unit 212 that emits ultraviolet light. White light or infrared light supplied from the light source unit 211 is guided to the distal end portion 205 through the insertion unit 202 and is emitted to the outside from the illumination window (for example, white light L_{WLI} illustrated in FIG. 12). In addition, ultraviolet light L_{UV} emitted from the UV light source unit 212 travels along the illumination optical path through the insertion unit 202 to be emitted to the UV absorption filter 208.

In the endoscope apparatus 200 described above, as in the first embodiment or the modifications, at the time of normal observation and infrared observation, in addition to white light or infrared light, ultraviolet light is emitted from the UV light source unit 212. As a result, the UV absorption filter 208 is irradiated with ultraviolet light. By the heat generation of the UV absorption filter 208 due to absorption of ultraviolet light, the temperature difference between the temperature of the body cavity and the temperature of the UV absorption filter 208 or the cover glass 2053 is reduced. As a result, the occurrence of dew condensation is suppressed.

As described above, even with the endoscope apparatus 200 including the flexible endoscope 201, the same effect as in the first embodiment described above can be obtained.

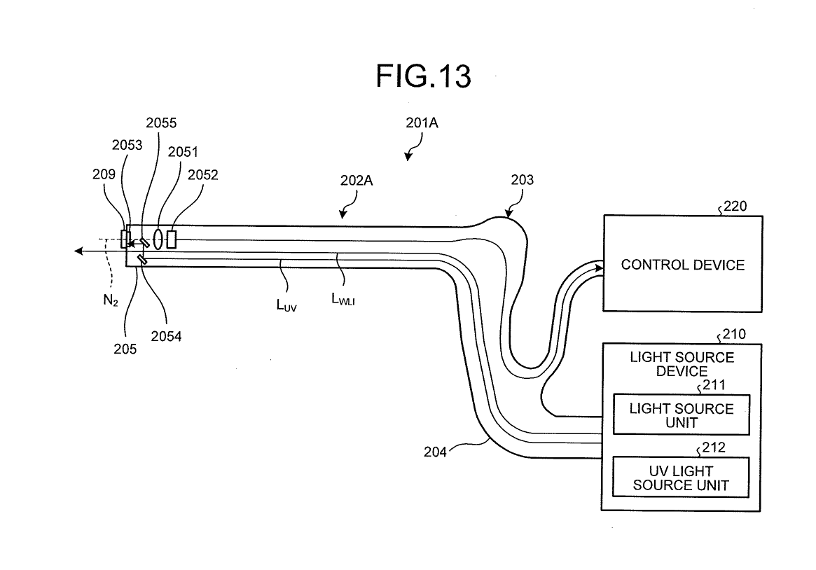

First modification of the second embodiment Next, a first modification of the second embodiment of the present disclosure will be described. FIG. 13 is a schematic diagram illustrating the configuration of an endoscope and a light source device according to the first modification of the second embodiment of the present disclosure. An endoscope apparatus according to the first modification is different from the endoscope apparatus 200 described above only in the arrangement of a UV absorption filter and a configuration for making ultraviolet light enter the observation optical system. The other configuration is the same as that of the endoscope apparatus 200 described above. Hereinafter, portions different from the above second embodiment will be described.

The endoscope apparatus according to the present first modification includes an endoscope 201A, a light source device 210, and a control device 220.

The endoscope 201A includes an insertion unit 202A that has flexibility and has an elongated shape, an operating unit 203 that is connected to the proximal end side of the insertion unit 202A and receives an input of various operation signals, and the above-described universal cord 204. In addition, the insertion unit 202A has the distal end portion 205, the bending portion 206 that can be bent, and the flexible tube portion 207 described above.

The insertion unit 202A further includes a first dichroic mirror 2054 and a second dichroic mirror 2055 at the distal end portion 205. The first dichroic mirror 2054 is provided on the optical path of illumination light, and reflects ultraviolet light and transmits light in a wavelength band other than the ultraviolet wavelength band. The second dichroic mirror 2055 is provided on the optical axis N₂ of the observation optical system, and reflects ultraviolet light and transmits light in a wavelength band other than the ultraviolet wavelength band.

In addition, the outer surface of the cover glass 2053 of the distal end portion 205 is covered with a UV absorption filter 209. The UV absorption filter 209 is a filter that absorbs light (ultraviolet light) in a wavelength band of 400 nm or less.

The ultraviolet light L_{UV} emitted from the UV light source unit 212 travels along the illumination optical path through the insertion unit 202A, is reflected by the first dichroic mirror 2054 and the second dichroic mirror 2055, and travels along the optical path of the observation optical system to be emitted to the UV absorption filter 209.

At the time of normal observation and infrared observation, in addition to white light or infrared light, ultraviolet light is emitted from the UV light source unit 212. As a result, the UV absorption filter 209 is irradiated with ultraviolet light. The UV absorption filter 209 generates heat by absorbing the ultraviolet light. By the heat generation of the UV absorption filter 209, the temperature difference between the temperature of the body cavity and the temperature of the UV absorption filter 209 or the cover glass 2053 is reduced. As a result, the occurrence of dew condensation is suppressed.

According to the first modification described above, as in the second embodiment, it is possible to suppress dew condensation at the distal end of the insertion unit 202 while suppressing an increase in the diameter of the insertion unit 202 and to perform observation using infrared light.

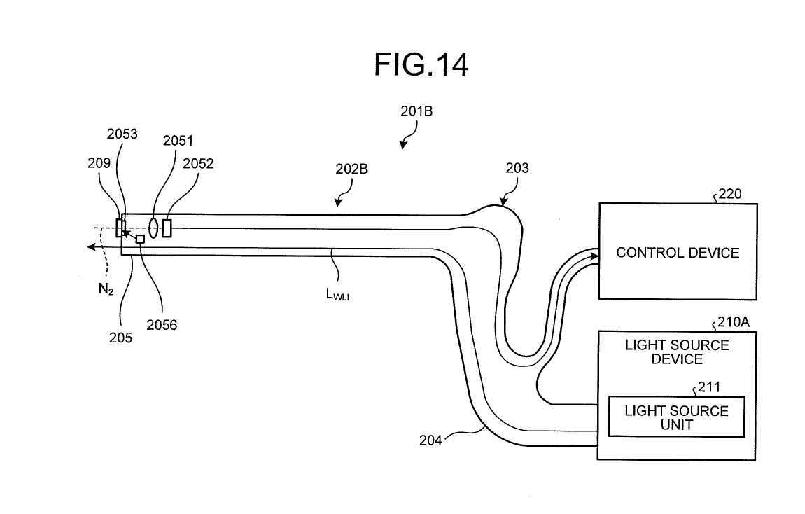

Second modification of the second embodiment Next, a second modification of the second embodiment of the present disclosure will be described. FIG. 14 is a schematic diagram illustrating the configuration of an endoscope and a light source device according to the second modification of the second embodiment of the present disclosure. An endoscope apparatus according to the second modification is different from the endoscope apparatus 200 described above only in the arrangement of a UV absorption filter and a UV light source unit. The other configuration is the same as that of the endoscope apparatus 200 described above. Hereinafter, portions different from the above second embodiment will be described.

The endoscope apparatus according to the present second modification includes an endoscope 201B, a light source device 210A, and a control device 220.

The endoscope 201B includes an insertion unit 202B that has flexibility and has an elongated shape, an operating unit 203 that is connected to the proximal end side of the insertion unit 202B and receives an input of various operation signals, and the above-described universal cord 204. In addition, the insertion unit 202B has the distal end portion 205, the bending portion 206 that can be bent, and the flexible tube portion 207 described above.

In addition, the outer surface of the cover glass 2053 of the distal end portion 205 is covered with a UV absorption filter 209. The UV absorption filter 209 is a filter that absorbs light (ultraviolet light) in a wavelength band of 400 nm or less.

The insertion unit 202B further includes a UV light source unit 2056 at the distal end portion 205. The ultraviolet light L_{UV} emitted from the UV light source unit 2056 is emitted to the UV absorption filter 209. By using an LED as the UV light source unit 2056, a small light source can be disposed at the distal end portion 205.

The light source device 210A includes a light source unit 211 capable of performing switching between the emission of white light and the emission of infrared light. White light or infrared light supplied from the light source unit 211 is guided to the distal end portion 205 through the insertion unit 202B and is emitted to the outside from the illumination window (for example, white light L_{WLI} illustrated in FIG. 14).

At the time of normal observation and infrared observation, in addition to white light or infrared light, the ultraviolet light is emitted from the UV light source unit 2056. As a result, the UV absorption filter 209 is irradiated with the ultraviolet light. The UV absorption filter 209 generates heat by absorbing the ultraviolet light. By the heat generation of the UV absorption filter 209, the temperature difference between the temperature of the body cavity and the temperature of the UV absorption filter 209 or the cover glass 2053 is reduced. As a result, the occurrence of dew condensation is suppressed.

According to the second modification described above, as in the second embodiment, it is possible to suppress dew condensation at the distal end of the insertion unit 202 while suppressing an increase in the diameter of the insertion unit 202 and to perform observation using infrared light.

Although the embodiments for carrying out the present disclosure have been described so far, the present disclosure should not be limited to the embodiments and modifications described above. In the embodiments described above, the description has been given on the assumption that the control device 5 performs signal processing and the like. However, the signal processing and the like may be performed on the camera head 9 side.

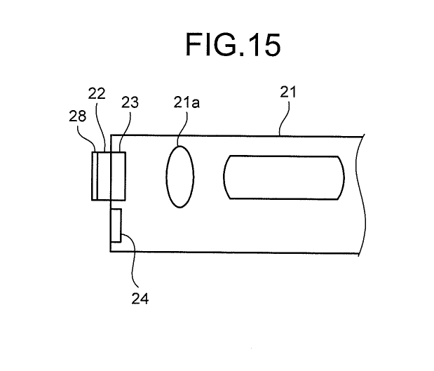

In addition, in the first and second embodiments and the modifications thereof described above, a hydrophilic coat may be provided on the outer surface of the UV absorption filter. FIG. 15 is a schematic diagram illustrating the distal end configuration of an endoscope according to another embodiment of the present disclosure. As illustrated in FIG. 15, a hydrophilic coat 28 may be provided on the outer surface of the UV absorption filter 22. By making the outer surface of the UV absorption filter hydrophilic, it is possible to suppress staying of the body fluid or the like on the outer surface.

As described above, the endoscope apparatus according to the present disclosure is useful for suppressing dew condensation at the distal end of the insertion unit while suppressing an increase in the diameter of the insertion unit and performing infrared observation using infrared light.

According to the present disclosure, it is possible to suppress dew condensation at the distal end of the insertion unit while suppressing an increase in the diameter of the insertion unit and to perform infrared observation using infrared light.

Although the disclosure has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. An endoscope apparatus, comprising:
an insertion unit having at a distal end thereof a light-incident end portion that captures observation light from a subject, the insertion unit being insertable into an object to be examined; and
an ultraviolet light source unit that emits ultraviolet light,
wherein the light-incident end portion is provided with an ultraviolet light absorption filter that generates heat by absorbing the ultraviolet light emitted from the ultraviolet light source unit.

2. The endoscope apparatus according to claim 1, further comprising:
an imaging unit that receives the observation light and generates an imaging signal;
an observation optical system that guides the observation light from the light-incident end portion to the imaging unit; and
at least one dichroic mirror provided in the observation optical system, the at least one dichroic mirror being configured to guide, to the imaging unit,
the ultraviolet light from the ultraviolet light source unit to the ultraviolet light absorption filter by one of transmission and reflection, and
at least light having a wavelength in a visible region in the observation light to the imaging unit by the other one of transmission and reflection.

3. The endoscope apparatus according to claim 2, further comprising:
an endoscope including the insertion unit, at least a part of the observation optical system, and the ultraviolet light absorption filter; and
a camera head including the imaging unit and the dichroic mirror, the camera head being detachably connected to the endoscope.

4. The endoscope apparatus according to claim 2,
wherein the at least one dichroic mirror is provided in the insertion unit.

5. The endoscope apparatus according to claim 1, further comprising an emission end portion from which illumination light for illuminating the object is emitted, the emission end portion being provided at the distal end of the insertion unit, wherein
the ultraviolet light absorption filter is attached to the light-incident end portion and the emission end portion.

6. The endoscope apparatus according to claim 1,
wherein a hydrophilic coat is formed on a surface of the ultraviolet light absorption filter.

7. The endoscope apparatus according to claim 1, further comprising:
a control unit that measures a temperature of the ultraviolet light absorption filter, based on an intensity of infrared light emitted from the ultraviolet light absorption filter, and controls an intensity of the ultraviolet light emitted from the ultraviolet light source unit according to a result of the measurement.

8. A medical imaging device detachably connected to an endoscope including an insertion unit having at a distal end thereof a light-incident end portion that captures observation light from a subject, the insertion unit being insertable into an object to be examined, the light-incident end portion having an ultraviolet light absorption filter that generates heat by absorbing ultraviolet light, the medical imaging device comprising:
an imaging unit that receives the observation light and generates an imaging signal; and
at least one dichroic mirror provided in an optical path of the observation light, the at least one dichroic mirror being configured to guide, to the imaging unit,
the ultraviolet light from the ultraviolet light source unit to the ultraviolet light absorption filter by one of transmission and reflection, and
at least light having a wavelength in a visible region of the observation light to the imaging unit by the other one of transmission and reflection.
